(19) United States
(12) Patent Application Publication
(10) Pub. No.: US 2012/0071451 A1
(43) Pub. Date: Mar. 22, 2012

(54) METHOD OF TREATING NONALCOHOLIC STEATOHEPATITIS WITH ELEVATED DOSES OF URSODEOXYCHOLIC ACID

(75) Inventors: Jean SPENARD, Mont-Saint-Hilaire (CA); Vlad RATZIU, Paris (FR); Marc RIVIERE, Saint Lambert (CA)

(73) Assignee: Aptalis Pharma Canada Inc., Mont-Saint-Hilaire (CA)

(21) Appl. No.: 13/217,042

(22) Filed: Aug. 24, 2011

Related U.S. Application Data
(63) Continuation of application No. PCT/IB2010/000551, filed on Mar. 16, 2010.
(60) Provisional application No. 61/160,955, filed on Mar. 17, 2009.

(51) Int. Cl. A61K 31/575; A61P 3/10; A61P 1/16

(52) U.S. Cl. 514/171; 514/182

(57) ABSTRACT

The present invention is directed to a method for the treatment of nonalcoholic steatohepatitis (NASH) by administering an elevated dose of ursodeoxycholic acid (UDCA), or a pharmaceutically acceptable salt thereof, to a patient in need of such treatment, wherein the patients demonstrate a significantly improved glycemic profile during treatment.

This application claims the benefit of U.S. Provisional Patent Application No. 61/160,955, filed Mar. 17, 2009, which is hereby incorporated by reference.

### FIELD OF THE INVENTION

The present invention relates to a method of treating nonalcoholic steatohepatitis (NASH) by administering an elevated dose of ursodeoxycholic acid (UDCA) to a patient in need thereof.

### BACKGROUND

The liver is the largest organ in the human body, located in the superior portion of the right upper abdomen. This organ is highly complex and specialized and performs many crucial biochemical functions. Critical liver functions involve the removal of toxins from the body and the manufacture of proteins related to energy storage and blood clotting. The liver is also involved in storing minerals, vitamins and glucose in the form of glycogen, which is metabolized in large quantities to provide energy, and also plays a role in red blood cell metabolism and the breakdown of certain metabolic byproducts in the blood stream.

NASH is a form of chronic liver disease often characterized by fibrosis. NASH sometimes progresses to cirrhosis and hepatocellular carcinoma, and may require liver transplantation in some patients. Patients suffering from NASH typically experience fatty deposits, tissue degeneration, inflammation, cell degeneration, cirrhosis, elevation of free fatty acids and other such abnormalities. NASH involves the development of histologic changes in the liver that are comparable to those induced by excessive alcohol intake but in the absence of alcohol abuse. Macrovesicular and/or microvesicular steatosis, lobular and portal inflammation, and occasionally Mallory bodies with fibrosis and cirrhosis characterize NASH. NASH is also commonly associated with hyperlipidemia, hyperglycemia, obesity, and type II diabetes mellitus. Obesity is the most common physiological condition that accompanies NASH, with approximately 70% or more of NASH sufferers displaying clinically diagnosed obesity. The extent of obesity in NASH patients tends to be generally correlated with the amount of steatosis and to be unrelated to non-insulin-dependent diabetes mellitis. However, non-insulin-dependent diabetes mellitis increases the prevalence of steatohepatitis especially in patients requiring insulin. Weight loss in patients before death does not appear to alleviate the steatosis and, somewhat paradoxically, obese patients who lost weight before death may actually have a higher incidence of steatohepatitis. The disease rarely occurs in any patient under the age of 30, but is particularly prevalent in patients in their 50s and 60s. Other clinical conditions characterized by steatohepatitis and inflammation include excessive fasting, jejunoileal bypass, total parental nutrition, chronic hepatitis C, Wilson's disease, and adverse drug effects such as those from corticosteroids, calcium channel blockers, high dose synthetic estrogens, methotrexate, and amiodarone.

The pathogenesis of NASH is unknown, but a correlation seems to exist between the degree of steatosis and the degree of fibrosis. See, e.g., Wanless et al., Hepatology, 12, 1106 (1990). Additionally, NASH may arise from the interaction of many different genes and life style factors. Mitochondrial impairment, oxidative stress and metabolic deregulation, have all been involved in the pathogenesis of steatohepatitis. Initial evaluation of patients suspected of NASH when present, are fatigue and right upper abdominal discomfort. Hepatomegaly is found in 90 percent of cases. Ultrasonography is currently the best method for detection of fatty infiltration of the liver. Elevated hepatocellular free fatty acids may cause membrane injury with subsequent inflammation, possible cholestasis, and subcellular organelle dysfunction. Cell death and fibrosis follow persistent inflammation, and cirrhosis occurs if the injury continues. Steatohepatitis is now considered an important cause of end-stage liver disease and may be the cause of an unknown number of cases of clyptogenic cirrhosis. See Powell et al., Hepatology, 11, 74 (1990). Unfortunately, once cirrhosis is established, the only therapeutic modality available is orthotopic liver transplantation.

NASH patients characteristically have normal to high levels of serum aminotransferases, such as aspartate aminotransferase (ASAT or AST) and alanine aminotransferase (ALAT or ALT) levels. ASAT levels may be higher than ALAT levels in patients with NASH. Gamma-glutamyl transpeptidase (Gamma-GT) levels are also typically elevated in NASH patients.

Since the prevalence of NASH-associated diseases (e.g., obesity and type II diabetes) is increasing, the prevalence of NASH is also expected to increase. Therefore, this disease has become an emerging public issue in the United States as well as in other countries. At present, there is no proven therapy for NASH. Since this disease affects mostly obese patients or patients with metabolic disease or diabetes, treatments for weight control and diabetes have been used in an effort to treat NASH and have shown some short term efficacy in improving liver condition. These treatments, however, are not without side effects or difficulties associated with their use. Thus, there remains an unmet need for a pharmacologic treatment with an excellent safety profile that provides long term liver protective therapy.

UDCA (also known as ursodiol) is a naturally occurring hydrophilic bile acid. UDCA is found in minute quantities in human bile and in larger quantities in the bile of certain species of bears. It is a bitter tasting white powder containing crystalline particles virtually insoluble in water but more soluble in intestinal fluids. UDCA is freely soluble in ethanol and glacial acetic acid, slightly soluble in chloroform, sparingly soluble in ether, and practically insoluble in water. UDCA is commercially sold under the trademarks URSO 250® and URSO Forte® for the treatment of patients with primary biliary cirrhosis. UDCA is also commercially sold under the trademark Actigall® for patients with gallbladder stones or for the prevention of gallstone formation in obese patients experiencing rapid weight loss.

UDCA is known for its heptaprotective characteristics (antiapoptotic, antioxidant, stabilizers of cell membranes) and immunomodulatory characteristics. UDCA has proven to be effective in certain chronic liver diseases where it was shown to improve liver function (Festi et al., Curr Clin Pharmacol 2(2):155-77 (May 2007)), and to result in the decrease of hydrophobic and potentially toxic bile acids (Angulo, Cur Gastroenterol Rep 4(1):37-44 (February 2002)).

In a small pilot study of NASH patients who received a one year treatment of 13-15 mg/kg/day UDCA, it was found that UDCA improved liver enzyme and steatosis levels, but did not change fibrosis or inflammation. Laurin et al., Hepatology 23(6):1464-67 (June 1996). In another study, the efficacy of two years of treatment with 13-15 mg/kg/day UDCA was evaluated in patients with NASH in a randomized, placebo-controlled trial. Lindor et al., Hepatology 39(3):770-78 (March 2004). The Lindor study did not show any differences between the UDCA treatment group and the placebo group. More recently, an open-label study by Georgescu and Georgescu, J Gastrointestin Liver Dis 16(1):39-46 (March 2007), assessed the efficacy of pentoxifylline, losartan, astorvastatin and UDCA in patients with NASH, and found that the 15 mg/kg/day UDCA treated group demonstrated a significant reduction in ALAT and Gamma-GT levels, but no improvements in steatosis, necroinflammation, or fibrosis. All of the above-mentioned studies were performed at a dose of 13-15 mg/kg/day, and none of them established UDCA as an adequate, effective therapy for NASH.

It has been reported that a dose response relationship exists with UDCA in patients suffering from primary biliary cirrhosis (van Hoogstraten et al., Aliment Pharmacol Ther 12(19):965-71 (October 1998)), primary sclerosing cholangitis (PSC) (Harnois et al., Am J Gastroenterol 96(5):1558-62 (May 2001); Mitchell et al., Gastroenterology 121(4):900-07 (October 2001)), and benign intrahepatic cholestasis of pregnancy (Mazzella et al, Hepatology 33(3):504-08 (March 2001)) as well as cystic fibrosis (van de Meeberg et al., Scand J Gastroenterol 32(4):369-73 (April 1997)). However, a more recently completed study of 28-30 mg/kg/day UDCA in adult PSC patients, which was conducted to assess the effects of UDCA on patient outcome and survival, concluded that UDCA could be related to a higher incidence of serious adverse events and poor overall outcomes, which could thus outweigh the biological improvements achieved with UDCA in PSC.

In this study, adult patients with PSC were enrolled in a randomized, double-blind controlled trial of 28-30 mg/kg/day UDCA versus placebo at seven different U.S. medical centers. More specifically, 150 adult patients with PSC were enrolled between 2002 and 2005 and treated with UDCA or placebo for up to 6 years. Patients underwent liver biopsy and cholangiography before therapy and at 5 years. Routine liver tests were performed every 3 months. Patients were assessed yearly, and endoscopy was performed at 2 and 5 years. The primary outcome measure was the development of hepatic decompensation, cholangiocarcinoma, liver transplantation, or death.

The study was terminated upon the recommendation of the Data Safety and Monitoring Board because of futility and concern over adverse effects. At enrollment, the UDCA (n=76) and placebo (n=74) groups were similar in respect to sex, age, duration of disease, serum ASAT and alkaline phosphatase (AP) levels, liver histology and Mayo risk score. During therapy, ASAT and AP levels decreased; the amount of decrease was greater for the UDCA than the placebo group (p<0.01). By the end of the study, 28 patients on UDCA (37%) versus only 17 patients on placebo (23%) had reached one of the pre-established clinical endpoints. When adjusted for baseline stratification characteristics (Mayo risk score, presence of gastroesophageal varices and histologic stage), the risk of a primary endpoint (i.e., death, liver transplant, minimal listing criteria for liver transplant, cirrhosis, esophageal and/or gastric varices, or cholangiocarcinoma) was 2.2 times greater for patients on UDCA than for those on placebo (p=0.011); for death or transplantation, the adjusted relative risk was 3.3 (p=0.029). The risk of reaching a primary endpoint was not modified by differences in age, gender, or presence of colitis. Serious adverse events were more common in the UDCA than placebo-treated groups (61% vs. 43%: p=0.03). The baseline Mayo risk score was strongly correlated with poor outcome as was the presence of cirrhosis on initial biopsy, but these effects were not different between the treatment groups.

This study concluded that 28-30 mg/kg/day UDCA therapy is associated with improvement in serum liver tests in PSC, but long term therapy does not improve survival, and may instead be associated with higher rates of serious adverse events and poor outcomes.

The present invention provides a new therapy regimen for NASH patients.

### SUMMARY OF THE INVENTION

The present invention relates to a method for treating NASH by administering a dose of about 28-35 mg ursodeoxycholic acid (UDCA), or a pharmaceutically acceptable salt thereof, per kg body weight per day to a patient in need thereof. In one embodiment, the method reduces fibrosis levels and/or liver inflammation levels in the patient compared to pre-treatment levels. In another embodiment, the glycemic index of the patient remains substantially stable during the treatment period. Suitable treatment periods may include 3 months, 6 months, 9 months, 12 months, 2 years, 3 years, 4 years, 5 years, etc., and longer. In one embodiment, the patients also suffer from type II diabetes. In another embodiment, the method further includes administration of an anti-diabetic drug, such as a thiazolidinedione.

### BRIEF DESCRIPTION OF THE DRAWINGS

FIG. 1 is a graph showing mean ALAT levels (IU/L) versus time in NASH patients receiving 30 mg/kg/day UDCA over one year as described in Example 2.

FIG. 2 is a graph showing mean change versus baseline for ALAT levels in NASH patients receiving 30 mg/kg/day UDCA over one year as described in Example 2.

FIG. 3 is a graph showing mean ASAT levels (IU/L) versus time in NASH patients receiving 30 mg/kg/day UDCA over one year as described in Example 2.

FIG. 4 is a graph showing mean change versus baseline for ASAT levels in NASH patients receiving 30 mg/kg/day UDCA over one year as described in Example 2.

FIG. 5 is a graph showing mean Gamma-GT levels (IU/L) versus time in NASH patients receiving 30 mg/kg/day UDCA over one year as described in Example 2.

FIG. 6 is a graph showing mean change versus baseline for Gamma-GT levels in NASH patients receiving 30 mg/kg/day UDCA over one year as described in Example 2.

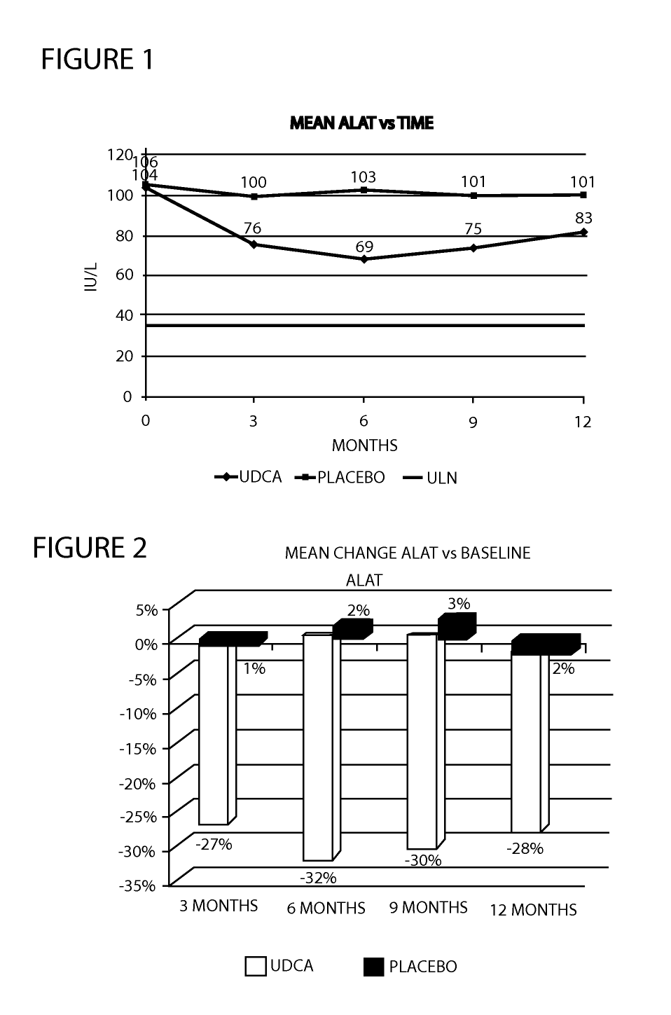

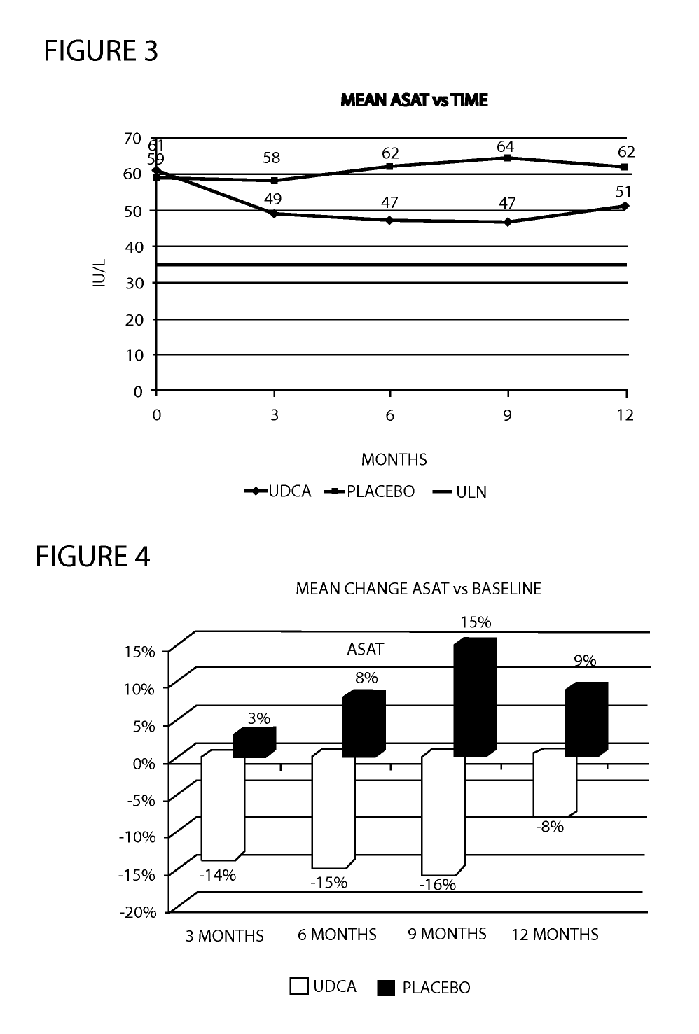

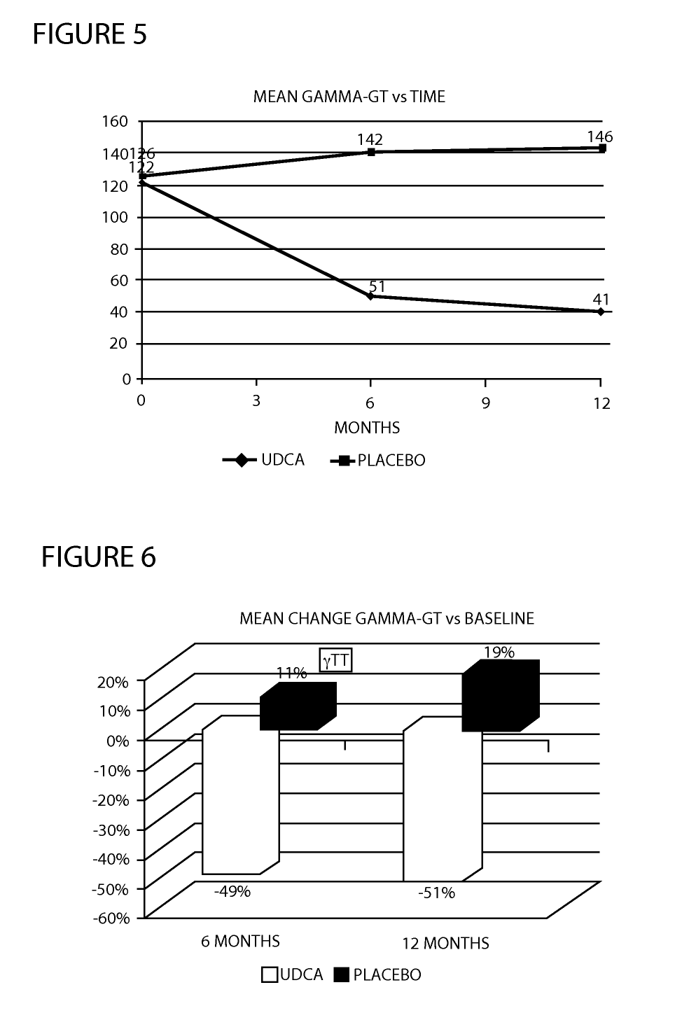

### DETAILED DESCRIPTION OF THE INVENTION

The present invention provides a method for treating NASH by administering 28-35 mg/kg/day UDCA. This method provides significant benefits to the patient including, for example, reduction of aminotransferase levels (e.g., ALAT and ASAT), reduction of Gamma-GT levels, reduced fibrosis, and reduced inflammation. Additionally, 28-35 mg/kg/day UDCA provides a significantly improved glycemic index to NASH patients treated by this method. In particular, NASH patients treated with 28-35 mg/kg/day UDCA according to the present invention experience stable levels of glycemia, insulinemia, and HbA1c, whereas NASH patients treated with a placebo have increased levels of glycemia, insulinemia, and HbA1c over time.

This is a surprising and highly beneficial effect of the present invention.

The chemical name of UDCA is 3α, 7β-dihydroxy-5β-cholan-24-oic acid). UDCA has the following molecular structure:

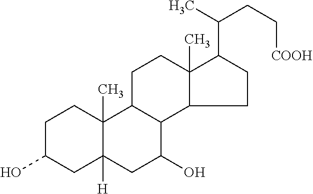

In accordance with the present invention, UDCA may be administered alone in its acid form or as a pharmaceutically acceptable salt thereof. All weights provided are based on the equivalent weight of the free acid unless otherwise specified. The present invention also includes pharmaceutical formulations that combine UDCA, or a pharmaceutically acceptable salt thereof, with one or more pharmaceutically acceptable carriers, excipients, diluents and/or additives, in either single or multiple doses. Such pharmaceutical formulations may be prepared in accordance with conventional techniques known to those skilled in the art.

In the present invention, a typical dosage of UDCA is in the range of about 28-35 mg/kg body weight per day (mg/kg/day), preferably about 28-30 mg/kg/day, more preferably about 30 mg/kg/day. The dosage may be administered as a single dose or may be divided into one or more doses, such as 2 to 6 doses per day, and preferably 2 to 4 doses per day. Preferably, the dosage of UDCA is administered daily, in the morning and in the evening. The exact dosage will depend upon the frequency and mode of administration, the sex, age, weight, and general condition of the subject treated, the nature and severity of the condition treated, the presence of any concomitant diseases to be treated concurrently, and other factors evident to those skilled in the art. Preferably, the dosage of UDCA is administered with food.

The pharmaceutical compositions of the present invention may be formulated to include other active ingredients—e.g., nutritional supplements such as vitamin E, anti-diabetic drugs such as sulfonylureas (e.g., tolbutamide, acetohexamide, tolazamide, chlorpropamide, glipizide, glyburide, glimepiride, and gliclazide), meglitinides (e.g., repaglinide and nateglinide), biguanides (e.g., metformin), alpha-glucosidase inhibitors (e.g., miglitol and acarbose), glucagons-like peptide (GLP) analogs and agonists (e.g., GLP-1, exenatide, exendin-4, and liraglutide), DPP-4 inhibitors (e.g., vildagliptin and sitagliptin), amylin analogs, PPARα and/or γ ligands (e.g., aleglitazar), sodium-dependent glucose transporter 1 (SGLT-1) inhibitors, fructose 1,6-bisphosphatase (FBPase) inhibitors, thiazolidinediones (including rosiglitazone, pioglitazone, troglitazone, and other glitazones), insulin, and other therapeutic agents. Suitable pharmaceutically acceptable carriers, excipients, diluents, and/or additives include, for example, vehicles, fillers, solvents, diluents, surfactants, colorants, preservatives, disintegrants, glidants, lubricants, flavours, binders, and wetting agents.

The pharmaceutical compositions of the present invention may be administered by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. The preferred route will depend on the general condition and age of the subject, and the nature of the condition to be treated.

The pharmaceutical compositions of the present invention may be formulated for oral administration as solid dosage forms, such as capsules, tablets, powders, and granules, and as liquid dosage forms, such as solutions, emulsions, suspensions, syrups, and elixirs. Where appropriate, solid dosage forms can be prepared with coatings such as enteric coatings or can be otherwise formulated so as to provide controlled or sustained release of the active ingredient according to methods that are well known in the art.

### EXAMPLES

The present invention is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled.

### Example 1

### UDCA Formulation

An example of a pharmaceutical composition of the present invention contains 250 mg UDCA (or 500 mg UDCA) in combination with the following inactive ingredients: microcrystalline cellulose, povidone, sodium starch glycolate, magnesium stearate, ethylcellulose, dibutyl sebacate, carnauba wax, hydroxypropyl methylcellulose, PEG 3350, PEG 8000, cetyl alcohol, sodium lauryl sulfate, and hydrogen peroxide. This pharmaceutical composition may be formulated as a film-coated tablet for oral administration.

### Example 2

### Clinical Study of 28-35 mg/kg/day UDCA for Treating NASH

A multicenter randomized double-blind placebo-controlled study was conducted to examine the efficacy and tolerability of 28-35 mg/kg/day UDCA in patients with histologically proven NASH, ALAT and/or ASAT greater than 50 IU/L. A total of 120 patients were planned to receive either UDCA or placebo for a period of 12 months. Treatment was administered with meals. During the study, liver biochemistry, tolerability and side effects were monitored regularly. During the study, overweight and obese patients were encouraged to lose weight by following a hypocaloric diet and to maintain a certain level of physical activity. Drug treatments taken by patients for associated medical conditions were allowed. At the end of the 12^{th} month, patients underwent an end of study evaluation and study treatments were stopped.

Study Population:

Inclusion criteria: age of patients higher than 18 years; liver biopsy compatible with NASH: presence of steatosis >20% associated with hepatocyte ballooning and/or hepatic lobule necrosis during the last 18 months; ALAT or ASAT levels >50 IU/L at the screening visit (with at least 3 elevated transaminase levels in the last 12 months)

Exclusion criteria: hepatic biopsy done before the last 18 months; no more than one normal value of transaminases during the last 12 months; patient was treated by UDCA during the last 12 months; loss of weight of more than 15% between the time of the hepatic biopsy and the screening; alcohol consumption higher than 20 g/day for women or higher than 30 g/day for men; presence of other causes of hepatitis such as chronic hepatitis B or C, increased serum ferritin associated with homozygosity for the C282Y mutation, primary biliary cirrhosis, primary sclerosing cholangitis, well documented autoimmune hepatitis (specific autoantibody, hpergammaglobulinaemia, histology-compatible), alpha-1 anti-trypsin deficiency, Wilson's disease, HIV infection; secondary causes of NASH: long term amiodarone-induced NASH, corticotherapy, obesity surgery within the last 2 years, Tamoxifen® treatment; Child's B or Child's C grade cirrhosis; presence of liver carcinoma; currently treated or treated during the past three years of hepatic biopsy with rosilitazone or pioglitazone; treatment with vitamin E during the past six months before screening; pregnant or breastfeeding women; unavailable histology slides for reading by the central laboratory.

Discontinuation criteria: Subjects were free to discontinue the study at any time for any or for no reason, and without prejudice to further treatment. Patients who withdrew subsequent to the pre-study evaluations but before receiving any study medication were not considered dropouts and were not included in the database. Patients who were included in the study and received at least one dose of study medication were included in the database and considered part of the safety population. Patients who were included in the study and received a dose of study medication and for whom at least one post-baseline evaluation was available were analyzed as part of the Intent-to-Treat (ITT) population. Patients from the ITT population who completed the study without any major protocol violations were analyzed as part of the Per Protocol (PP) population.

Dropouts might have occurred because of the following reasons, among others: the patients had been included in violation of the inclusion/exclusion criteria; the patient chose to discontinue participation for personal reasons (moving away, no time, etc.); the sponsor discontinued the patient following an adverse event; the investigator or the sponsor discontinued the patient for a significant protocol violation; the patient used a prohibited medication during the study; the patient developed and immediate medical condition or required a surgical procedure that would have compromised the patient's continued participation and was discontinued from the study.

The study treatment was to be stopped in the following instances: an increase in liver transaminases 5 times higher than the pre-study levels (There is no reported hepatotoxicity associated with UDCA except in rare cases of decompensated liver cirrhosis. There usually exists a fluctuation in liver transaminases in NASH and only a 5 times increase rather than a 3 times increase from the pre-study levels would require the cessation of study medication.);

occurrence of cutaneous allergic reactions.

Main Endpoints: The primary endpoint was percent change in ALAT at 12 months vs. baseline. The secondary endpoints included: percent change in ASAT at 12 months vs. baseline; percent change Gamma-GT at 12 months vs. baseline; percent pts with normalized ALAT at 12 months; percent pts with normalized ASAT at 12 months; change in fibrosis index (FibroTest); change in inflammation index (Actitest); change in metabolic syndrome markers; and safety.

FibroTest is a non-invasive blood test that provides a quantitative estimate of liver fibrosis and can be used to predict advanced fibrosis. ActiTest is a non-invasive blood test that is used to assess the activity of liver disease by measuring the degree of necrosis and inflammation.

Patient demographics are summarized in Table 1.

TABLE 1

| | | UDCA | Placebo |
|---|---|---|---|
| N | | 62 | 64 |
| Mean age (SD) | years | 49.8 (10.2) | 49.6 (12.6) |
| Gender | M | 75.8% | 75.0% |
| Mean height (SD) | cm | 170.5 (9.5) | 172.3 (9.1) |
| Mean weight (SD) | kg | 89.5 (14.8) | 91.8 (17.1) |
| Smokers | yes | 21.0% | 10.9% |

Metabolic Syndrome Markers are summarized in Table 2.

TABLE 2

| | | UDCA | Placebo |
|---|---|---|---|
| N | | 62 | 64 |
| Non-Insulin-Dependent | yes | 24 (39%) | 16 (25%) |
| Diabetes Mellitus | | | |
| Arterial hypertension | yes | 30 (48%) | 20 (31%) |
| Dyslipidemia | yes | 36 (58%) | 32 (52%) |
| Hypercholesterolemia | yes | 26 (42%) | 28 (44%) |
| Hypertriglyceridemia | yes | 24 (39%) | 19 (30%) |

Treatments: UDCA was provided at a dose of 30 mg/kg/day, taken in two divided doses with meals—once in the morning, and once in the evening. Placebo tablets (excipient without active compounds) were prepared to have a similar appearance as UDCA tablets to ensure the double-blindness. Placebo tablets were also taken in the same divided doses like UDCA tablets.

Methods—Assigning patients to treatment groups, selecting doses, and selecting timing of dose for each patient: Patients were to be randomized in a 1:1 (active:placebo) proportion. The use of placebo was to ensure the double-blindedness of the trial. There was no a priori stratification planned. Randomization was in blocks of four (two for UDCA and two for placebo). As per its label, the drug has to be administered in 2-4 divided doses with food. In the present study, the doses used were 30 mg/kg/day, and the dose for each patient depended on patient weight.

Efficacy and Safety: Efficacy evaluations included measurements of serum transaminase levels as well as serum markers of fibrosis. Patients with elevated levels of serum transaminases and with a liver biopsy indicative of NASH (>20% steatosis associated with hepatic ballooning and/or intralobular necrosis (Brunt et al., Am J Gastroenterol 94(9):2467-74 (September 1999)) were eligible for the study. The liver biopsy should date from less than 18 months in patients with stable metabolic condition (no recent weight loss, no recent (in the last 6 months) antidiabetic treatment with metformin, sulfonamides or insulin). Four original slides and/or six blank slides (i.e., non-colored) of the liver biopsy were to be reviewed by a pathologist. For homogeneous centralized reading of the slides, the latter were colored by Haematoxylin-eosin staining, Hemalun Sirius Red staining, and Perls staining for confirmation of the histologic entry criteria. Only after the informed consent signature and confirmatory histology were the blood tests performed.

The non-invasive measure of hepatic fibrosis was done by measuring the serum levels of apolipoprotein A1, total bilirubin, Gamma-GT, alpha-2 microglobulin, haptoglobulin, and ALAT with calculation of the FibroTest and ActiTest score. Measurement of hyaluronic acid, carbohydrate-deficient transferrin (CDT), and transferrin were also performed according to Laine et al., Hepatology 39(6):1639-46 (June 2004). Insulin resistance was measured biologically using the simplified Homeostasis Model Assessment (HOMA-IR) that takes into account glucose levels and fasting blood sugar. Clinical evaluation of insulin resistance was based on waist circumference measurements (due to its associating with visceral adiposity) and on the calculation of the body mass index (BMI)=weight (kg)/height (m²).

Results: See Tables 3-7.

TABLE 3
Mean change at 12 months vs. baseline (all subjects)

| | UDCA | Placebo | p |
|---|---|---|---|
| ITT | n = 62 | n = 64 | |
| Δ % glycemia (SD) | −1% (21) | +11% (24) | p = 0.023 |
| Δ % insulinemia (SD) | −5% (59) | +204% (1357) | p = 0.038 |
| Δ % HbA1c (SD) | −1% (10) | +8% (15) | p < 0.05 |
| Δ % HDL (SD) | −1.4% (20) | −1.4% (16) | p = 944 |
| Δ % LDL (SD) | −6.2% (26) | −1% (18) | p = 0.285 |
| Δ % Total cholesterol (SD) | −4.1% (19) | −0.6% (11) | p < 0.336 |
| Δ % Triglycerides (SD) | +5.6% (37) | +13.3% (41) | p < 0.294 |

Effects on Glycemia (metabolic syndrome): Glycemia increased in the placebo group, but remained stable in the UDCA treatment group. This is a statistically significant difference (p=0.023). Insulinemia was lower in the UDCA treatment group at 12 months (p=0.038). HbA1c was lower in the UDCA treatment group at 6 and 12 months (p<0.05).

TABLE 4
Mean change at 12 months vs. baseline

| | UDCA | Placebo | |
|---|---|---|---|
| | n = 62 | n = 64 | |
| ITT | | | |
| Δ % ALAT (SD) | −28% (55) | −2% (35) | p < 0.001 |
| Δ % ASAT (SD) | −8% (59) | +9% (37) | p < 0.001 |
| Δ % Gamma-GT (SD) | −51% (28) | +19% (48) | p < 0.001 |
| Normalization ALAT | 25% | 5% | p = 0.003 |
| Normalization ASAT | 32% | 23% | p = 0.253 |
| PPP | | | |
| Δ % ALAT (SD) | −23% (59) | +0.8% (37) | p < 0.001 |
| Δ % ASAT (SD) | −3% (63) | +11% (39) | p = 0.007 |
| Δ % Gamma-GT (SD) | −49% (29) | +19% (48) | p < 0.001 |
| Normalization ALAT | 29% | 6% | p = 0.004 |
| Normalization ASAT | 36% | 18% | p = 0.048 |

Effects on Liver Enzymes: The percent change from baseline ALAT is significant at 3, 6, and 9 months, with the maximum effect seen at 3 months. The percent change from baseline ASAT is significant at 3, 6, 9, and 12 months, with the maximum effect seen at 3 months. The percent change from baseline in Gamma-GT is significant at 6 and 12 months, with the maximum effect seen at 6 months.

TABLE 5
Individual effect on fibrosis

| | Fibrosis | | | |
|---|---|---|---|---|
| Treatment | Increased | Stable | Decreased | Total |
| N (UDCA) | 5 | 31 | 20 | 56 |
| | 8.9% | 55.4% | 35.7% | |
| | p = 0.0038 | NS | p = 0.0116 | |
| N (Placebo) | 18 | 32 | 9 | 59 |
| | 30.5% | 54.2% | 15.3% | |

TABLE 6
FibroTest (effect on fibrosis) change vs. baseline

| | UDCA | p | Placebo |
|---|---|---|---|
| ITT | | | |
| At 6 months | | | |
| N | 58 | | 62 |
| Mean | −9.15% | 0.006 | 6.13% |
| SD | 29.84% | | 38.27% |
| At 12 months | | | |
| N | 53 | | 62 |
| Mean | −8.11% | <0.001 | 20.65% |
| SD | 35.37% | | 45.06% |
| PP | | | |
| At 6 months | | | |
| N | 42 | | 50 |
| Mean | −9.68% | 0.002 | 9.99% |
| SD | 30.63% | | 39.77% |
| At 12 months | | | |
| N | 42 | | 51 |
| Mean | −5.40% | <0.001 | 22.13% |
| SD | 36.94% | | 46.98% |

As shown in Table 6, the patients in the UDCA treatment group (both the ITT and the PP populations) showed significant improvement in fibrosis levels as compared to patients in the placebo group.

TABLE 7
ActiTest (effect on liver inflammation) change vs. baseline

| | UDCA | p | Placebo |
|---|---|---|---|
| ITT | | | |
| At 6 months | | | |
| N | 58 | | 62 |
| Mean | −31.41% | <0.001 | −1.04% |
| SD | 32.06% | | 34.12% |
| At 12 months | | | |
| N | 53 | | 62 |
| Mean | −30.97% | <0.001 | −2.90% |
| SD | 35.59% | | 33.92% |
| PP | | | |
| At 6 months | | | |
| N | 42 | | 50 |
| Mean | −31.23% | 0.002 | 1.17% |
| SD | 33.56% | | 33.99% |
| At 12 months | | | |
| N | 42 | | 51 |
| Mean | −27.33% | <0.001 | −1.61% |
| SD | 37.25% | | 35.10% |

As shown in Table 7, the patients in the UDCA treatment group (both the ITT and the PP populations) showed significant improvement in liver inflammation levels as compared to patients in the placebo group.

Safety Results: GI symptoms (diarrhea, abdominal pain, motility problems) were more frequent (−3×) in the UDCA treatment group than in the placebo group. RUQ pain and asthenia were more prevalent (−2×) at entry in the UDCA treatment group than in the placebo group, but the difference disappears at 3 months.

Summary: A total of 126 patients (64 placebo and 62 UDCA) were enrolled (ITT population) in the study. There were 75% males, mean age (±SD) was 49.7±11.5 years, and BMI (±SD) was 30.9±5.1 kg/m². Metabolic syndrome, hypertension, and type-II diabetes were present in 40%, 32%, and 35% of the patients, respectively. After 12 months, ALAT decreased by (mean±SD) −28±55% in the UDCA treatment group compared to −2±35% in the placebo group, respectively (p=0.003). Mean (±SD) decreases in serum ASAT and Gamma-GT levels for the UDCA treatment group were −8±59% and −51±28%, respectively; compared to placebo where these factors increased by +9±37% (p<0.001) and +19±48% (p<0.001), respectively. All results were confirmed in the PP population. Asthenia and right upper quadrant pain (RUQP) were reported more frequently at baseline in the UDCA treatment group than in the placebo group. This difference disappeared early during treatment (3 months). Changes in serum markers of insulin resistance, fibrosis, inflammation and apoptosis are reported. The UDCA treatment group experienced more mild diarrhea, abdominal pain, and gastrointestinal motility disorders than the placebo group.

Conclusion: This randomized, controlled trial demonstrated a significant and marked biochemical response to 28-35 mg/kg/day UDCA treatment in NASH patients and suggested symptomatic improvement of asthenia and RUQP, without any significant safety concerns.

All references cited and/or discussed in this specification are incorporated herein by reference in their entireties and to the same extent as if each reference was individually incorporated by reference.

1. A method for treating nonalcoholic steatohepatitis (NASH), comprising administering a dose of about 28-35 mg ursodeoxycholic acid (UDCA), or a pharmaceutically acceptable salt thereof, per kg body weight per day to a patient in need thereof,
wherein the method reduces fibrosis levels and/or liver inflammation levels in the patient compared to pre-treatment levels.

2. The method of claim 1, wherein the dose is about 28-30 mg/kg/day.

3. The method of claim 1, wherein the dose is administered as a single daily dosage.

4. The method of claim 1, wherein the dose is administered in 2-4 divided dosages per day.

5. The method of claim 1, wherein the glycemic index of the patient remains substantially stable during the treatment period.

6. The method of claim 1, wherein the treatment is provided for a period of at least 6 months.

7. The method of claim 6, wherein the treatment is provided for a period of at least 12 months.

8. The method of claim 1, wherein the UDCA is administered with food.

9. The method of claim 1, wherein the UDCA is administered daily in the morning and the evening.

10. The method of claim 1, wherein the patient suffers from type II diabetes.

11. The method of claim 1, further comprising administering an anti-diabetic drug.

12. The method of claim 11, wherein the anti-diabetic drug is a thiazolidinedione.

13. The method of claim 2, wherein the UDCA is administered with food.

14. The method of claim 2, wherein the UDCA is administered daily in the morning and the evening.

15. The method of claim 2, wherein the patient suffers from type II diabetes.

16. The method of claim 2, further comprising administering an anti-diabetic drug.

17. The method of claim 16, wherein the anti-diabetic drug is a thiazolidinedione.

18. The method of claim 2, wherein the treatment is provided for a period of at least 6 months.

19. The method of claim 13, wherein the treatment is provided for a period of at least 6 months.

20. The method of claim 15, wherein the treatment is provided for a period of at least 6 months.
